(19) United States
(12) Patent Application Publication
(10) Pub. No.: US 2026/0201433 A1
(43) Pub. Date: Jul. 16, 2026

(54) CHEMOENZYMATIC SYNTHESIS OF POLYKETIDES

(71) Applicant: Aspera Biomedicines, Inc., La Jolla, CA (US)

(72) Inventors: Catriona H. M. Jamieson, La Jolla, CA (US); James J. La Clair, La Jolla, CA (US); Michael D. Burkart, San Diego, CA (US)

(21) Appl. No.: 19/134,662

(22) PCT Filed: Dec. 1, 2023
(86) PCT No.: PCT/US2023/082140

§ 371 Date: May 30, 2025

Related U.S. Application Data
(60) Provisional application No. 63/429,356, filed on Dec. 1, 2022.
(60) Provisional application No. 63/432,011, filed on Dec. 12, 2022.

(51) Int. Cl. C12P 17/02; A61K 31/365; C07D 303/02; C07D 317/30; C07D 407/06; C12N 9/04; C12P 17/04; C12P 17/16

(52) U.S. Cl. CPC C12P 17/02; A61K 31/365; C07D 303/02; C07D 317/30; C07D 407/06; C12N 9/0006; C12P 17/04; C12P 17/16; C12Y 101/01

(57) ABSTRACT

Provided herein are synthetic methods and intermediates useful in the preparation of polyketides. The polyketides include 12-membered ring macrolides. These synthetic methods and intermediates provide scalable access to polyketides, which include polyketides having potent bioactive splice modulator activity and potential for therapeutic applications including treating diverse types of cancers.

### RELATED APPLICATIONS

This application claims priority of U.S. Provisional Patent Application No. 63/432,011, filed Dec. 12, 2022, and U.S. Provisional Patent Application No. 63/429,356, filed Dec. 1, 2022, the entire content of each of which are incorporated herein by reference.

### SUMMARY

The double-stranded RNA-specific adenosine deaminase family of enzymes is encoded by the ADAR family genes. ADAR (adenosine deaminase acting on RNA) proteins enzymatically performs base editing, e.g., converts adenosine to inosine, which subsequently disrupts the otherwise orthogonal Watson-Crick pairing.

Unlike therapeutic use of CRISPR-Cas9, which acts at the gene editing level and is therefore a permanent or inheritable intervention, ADAR enzymes act on double-stranded RNA, which is a transient molecule. Therefore, ADAR modulation has potential as a transient and tunable, non-heritable, therapeutic intervention. ADAR enzymes have previously been referred to as double-stranded RNA adenosine deaminase (dsRAD; “Toward the therapeutic editing of mutated RNA sequences,” PNAS, 1995, 92, 8298-8302). Rather than using the CRISPR-Cas9 gene editing approach, which risks permanent genetic off-target mutations, RNA editing allows changes to the coding for protein production to be modulated without permanent gene editing. Accordingly, there is a continuing and long standing need for therapeutic intervention at the ribonucleic acid-to-protein stage of molecular biology's central dogma, including a need for modulators of the spliceosome and ADAR enzymes.

Thus, provided herein are commercially scalable synthetic methods and intermediates useful in the preparation of polyketides. The polyketides include 12-membered ring macrolides, including Compound A, Compound B, and pladienolides (including pladienolides A, B, C, D, E, F, and G). These compounds modulate the spliceosome and have shown the ability to down-regulate levels of ADAR enzymes.

### BRIEF DESCRIPTION OF THE DRAWINGS

FIG. 1 shows a synthetic scheme, including a chemoenzymatic transformation, for preparing Compound 14, which is an intermediate useful for preparing polyketides, including Compound A.

FIG. 2 shows a synthetic scheme, including a chemoenzymatic transformation, for preparing Compound A, which includes combining Compound 14 and Compound 20 to prepare Compound A.

### DETAILED DESCRIPTION

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “alkyl” refers to a saturated hydrocarbon, which may include linear, branched, or cyclic saturated hydrocarbons, or a mixture thereof.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The term “aryl” refers to a carbocyclic aromatic system comprising one, two, three, or more rings.

The term “composition” refers to a mixture of at least two or more components.

The modifier “C_{x-y}” refers to a moiety comprising x to y carbon atoms, wherein x and y are, independently, integers.

The terms “effective amount” and “therapeutically effective amount” refer to an amount of therapeutic compound, such as a compound prepared as described herein, including Compound A, Compound B, Pladienolide B, or the like, administered to a subject, either as a single dose or as part of a series of doses, which is effective to produce a desired therapeutic effect. In general, the therapeutically effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions described herein are known in the art and described, for example, in “Remington's Pharmaceutical Sciences” (Genaro (Ed.), Mack Publishing Co., 1985), the entire content of which is incorporated herein by reference.

The term “pharmaceutical composition” refers to a mixture of at least one compound described herein with a pharmaceutically acceptable carrier. The pharmaceutical composition facilitates administration of the compound to a patient or subject. Multiple techniques of administering a compound exist including, but not limited to, intravenous, oral, aerosol, parenteral, ophthalmic, pulmonary, and topical administration.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability. When any such inclusion or exclusion occurs, the specification is deemed to contain the group as modified thus fulfilling the written description of all Markush groups used in the appended claims.

References have been made to patents and printed publications throughout this specification, each of which are individually incorporated herein by reference in their entirety.

It is to be understood that the embodiments of this disclosure are illustrative. Accordingly, the present disclosure is not limited to that precisely as shown and described.

### Synthetic Methods and Intermediates

A number of synthetic schemes and intermediate compounds have been discovered, which are useful in improving the commercial synthetic preparation of polyketides, including Compound A. Compound A has been described, including in WO 2021/026273 A1 and U.S. Pat. No. 10,675,267 B2, which are incorporated herein by reference. Compound A has also been synthesized as described by Chan et al. (Cell Reports Physical Science, 2020, 1, 12, 100277).

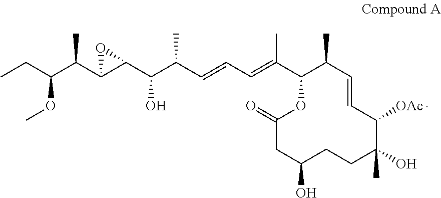 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]

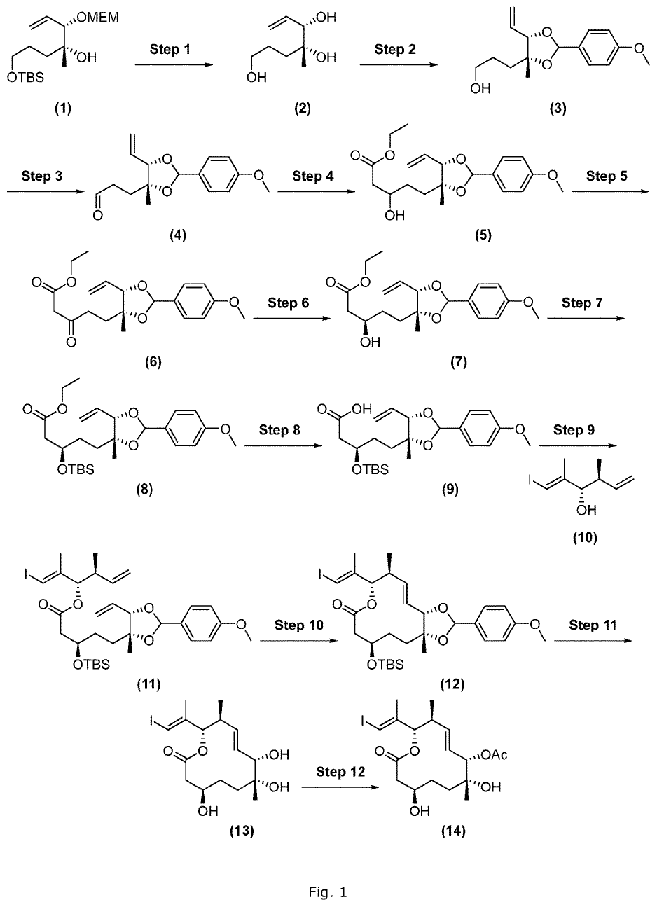

In some embodiments, Step 1, Step 2, Step 4, Step 5, Step 6, Step 7, Step 8, Step 16, Step 17, Step 4a, Step 5a, Step 6a, Step 7a, Step 3b, Step 4b, Step 5b, or a combination thereof, as shown in FIGS. 1-4, have been discovered as new and useful in preparation of polyketides such as Compound A. Additionally, in some embodiments, Compound 2, Compound 5, Compound 6, Compound 7, Compound 8, Compound 19-TPS, Compound 19R-TPS, Compound 190, Compound 20, Compound 21, Compound 22, Compound 23, Compound 24, Compound 25, and Compound 26 have been discovered as new and useful in preparation of polyketides such as Compound A.

In some embodiments, the schemes and intermediates provided herein may be adjusted by one of skill to prepare polyketides other than Compound A, including Compound B or stereoisomers thereof, pladienolides or stereoisomers thereof, such as pladienolide B, and compounds of the same class of polyketides that are capable of binding to the SF3b complex of a spliceosome, inhibiting mRNA splicing activity, or down-regulating ADAR levels. Pladienolide B has been synthesized previously, at least, as described, for example, by Rhoades et al. (Journal of the American Chemical Society 2021 143 (13), 4915-4920 DOI: 10.1021/jacs.1c01135).

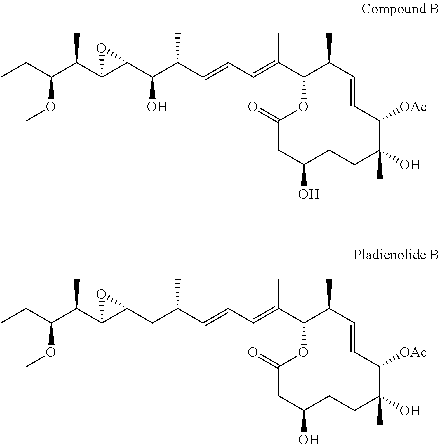 [SMILES: CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C ; CC[C@H](OC)[C@@H](C)[C@H]1O[C@@H]1C[C@H](C)/C=C/C=C(\C)[C@H]1OC(=O)C[C@H](O)CC[C@@](C)(O)[C@@H](OC(C)=O)/C=C/[C@@H]1C]

Compounds described herein also include isotopically-labeled compounds wherein one or more atoms is replaced by an atom having the same atomic number, but an atomic mass or mass number different from the atomic mass or mass number predominantly found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, or ¹⁸O. In some embodiments, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

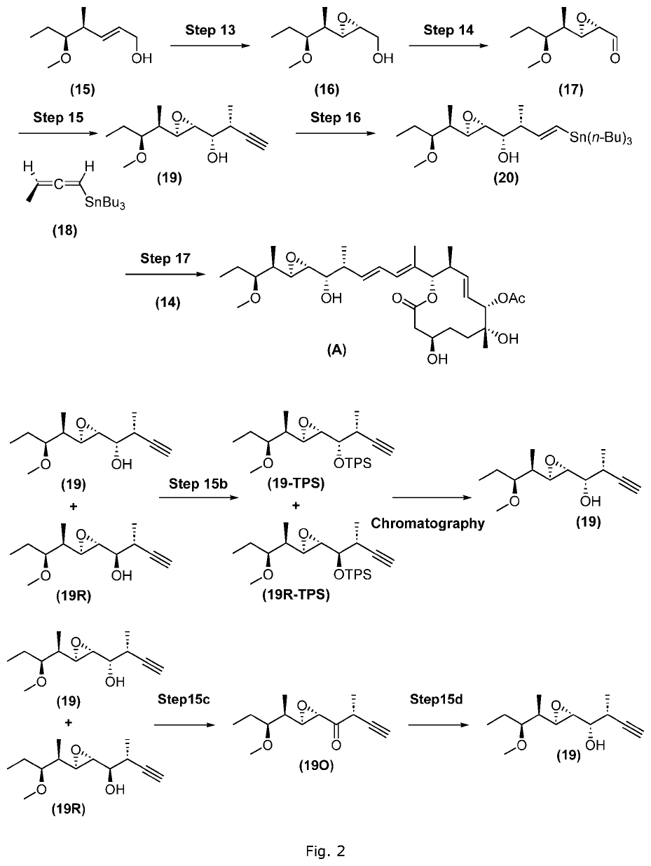

In some embodiments, the compounds described herein may be prepared by a method of synthesis that comprises any of the synthetic steps described in the Examples or in FIG. 1, FIG. 2, FIG. 3, or FIG. 4 pertinent to the compound being prepared.

In some embodiments, provided herein is a compound, selected from:

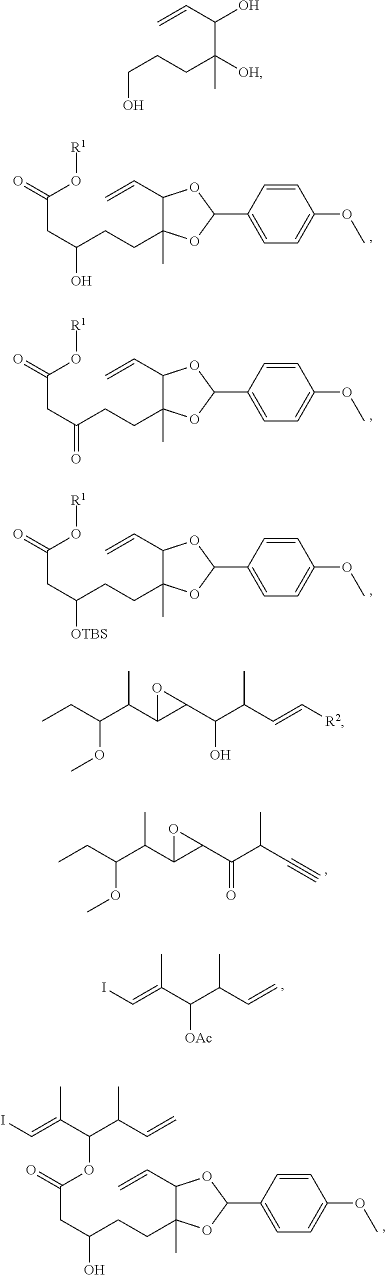

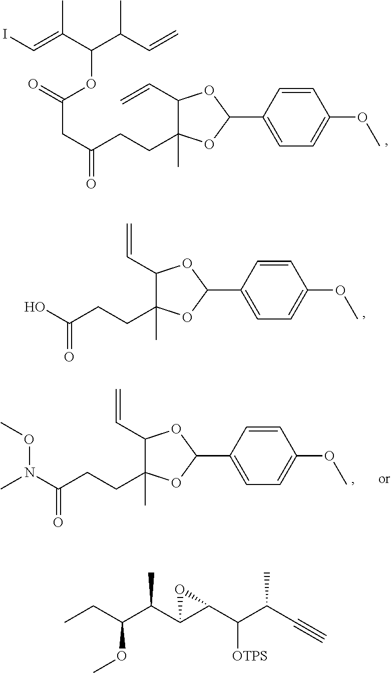

or a salt thereof,
wherein
R¹ is C₁₋₆ alkyl (e.g., ethyl), and
R² is an organotin moiety including a Tin (Sn) atom covalently linked with the carbon to which R² is attached (e.g., Sn(C₁₋₆ alkyl)₃, e.g., Sn(n-Bu₃) or SnMe₃).

In some embodiments, the compound is selected from:

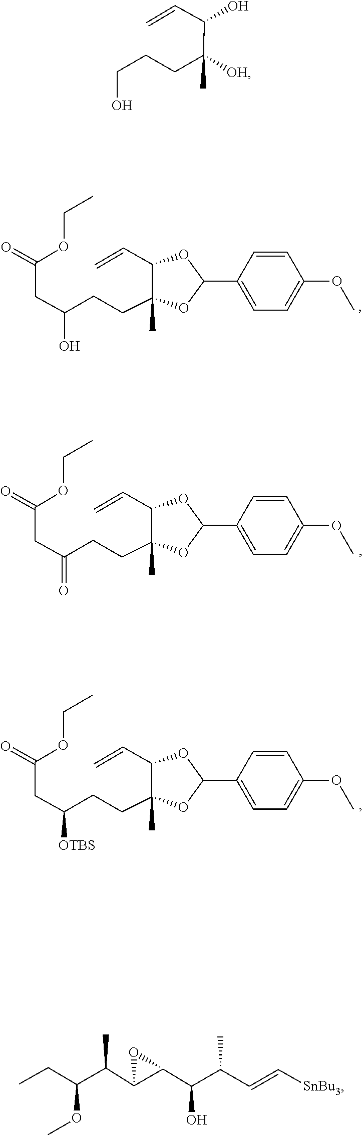

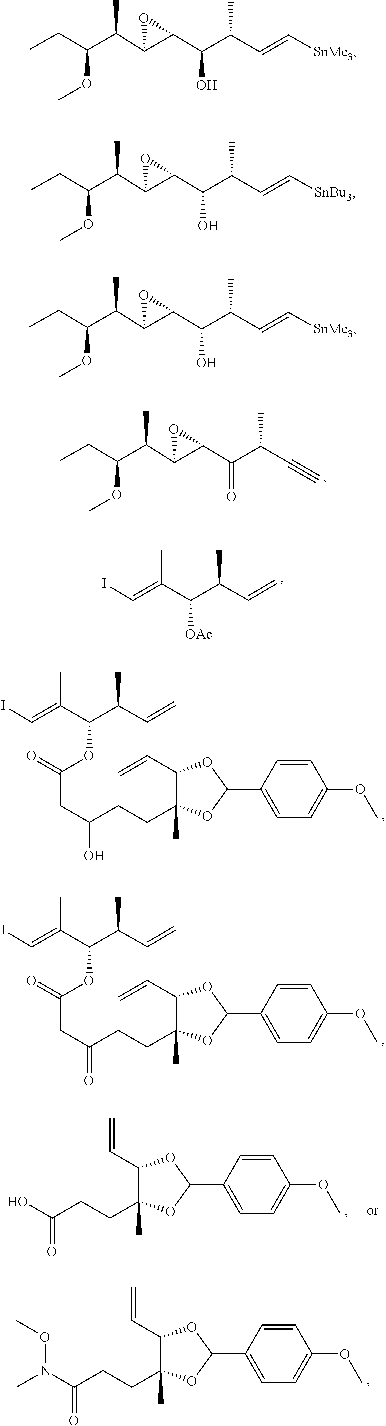

or a salt thereof.

In some embodiments, the compound is selected from:

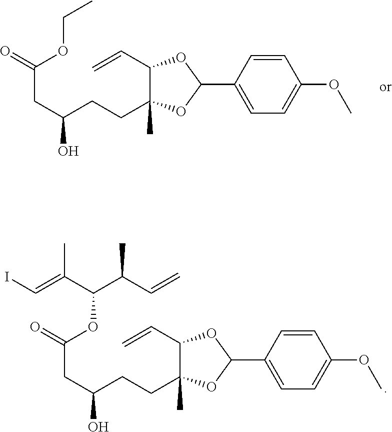

In some embodiments, still further compounds useful in preparing the pladienolides herein are compounds selected from:

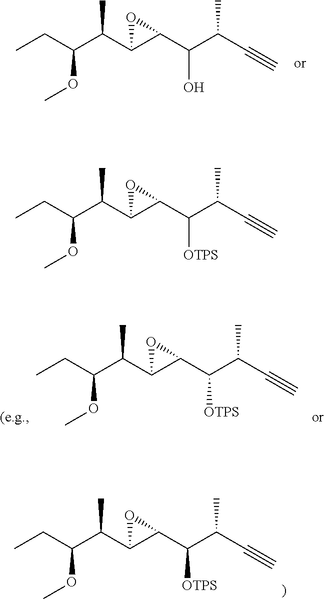

wherein TPS is triphenylsilanyl.

In some embodiments, provided herein are compositions, comprising a compound provided herein.

In some embodiments, the compounds or compositions provided herein are for use in the preparation of a synthesized compound.

In some embodiments, provided herein are methods of preparing a synthesized compound, comprising contacting a compound provided herein with one or more reagents to form the synthesized compound.

In some embodiments, provided herein are methods of preparing a compound provided herein, comprising contacting a precursor compound with one or more reagents to form the compound provided herein.

In some embodiments, provided herein are methods of preparing a synthesized compound, comprising at least one of the following steps:
Step 1) preparing a mixture comprising at least one solvent, 10-camphorsulfonic acid, and

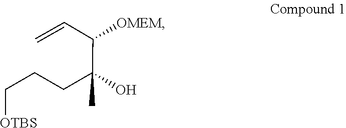

to form Compound 2

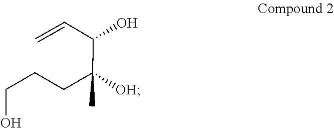

Step 2) preparing a mixture comprising at least one solvent, an acid, 1-(dimethoxymethyl)-4-methoxy-benzene, and Compound 2

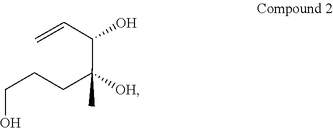

to form Compound 3

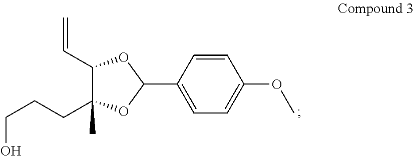

Step 4) preparing a mixture comprising at least one solvent, lithium diisopropylamide, and Compound 4

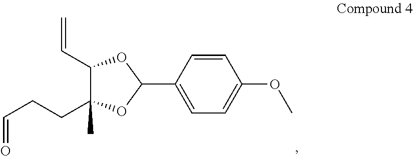

to form Compound 5

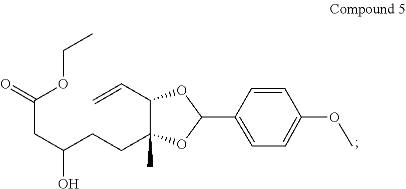

Step 5) preparing a mixture comprising at least one solvent, 2-iodoxybenzoic acid, and Compound 5

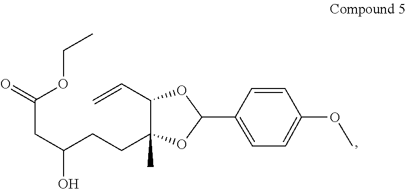

to form Compound 6

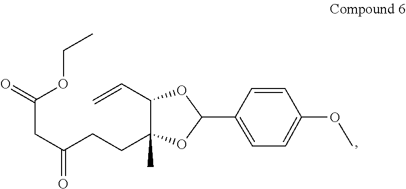

or
preparing a mixture comprising at least one solvent, N,N′-dicyclohexylcarbodiimide, pyridine·trifluoroacetic acid, and Compound 5

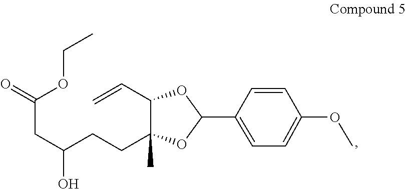

to form Compound 6

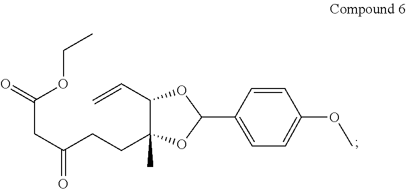

Step 6) preparing a mixture comprising at least one solvent, an enzyme having aldo-keto reductase activity, a reducing cofactor selected from NADPH or NADH, an auxiliary enzyme and substrate for cofactor regeneration, and Compound 6

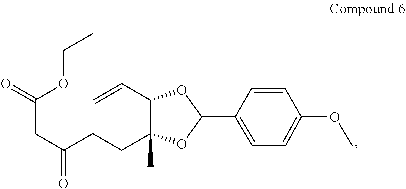

to form Compound 7

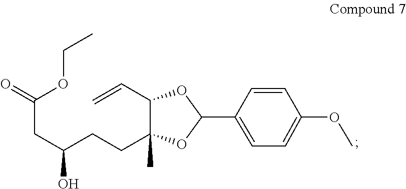

Step 7) preparing a mixture comprising at least one solvent, tert-butyldimethylsilyl chloride, an imidazole, tert-butyldimethylsilyl trifluoromethanesulfonate, a base, and Compound 7

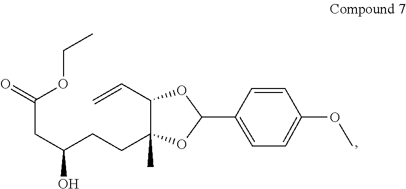

to form Compound 8

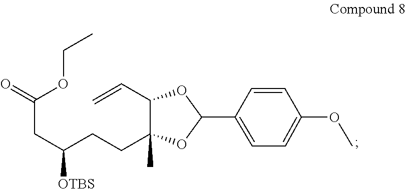

Step 8) preparing a mixture comprising at least one solvent, a base, and Compound 8

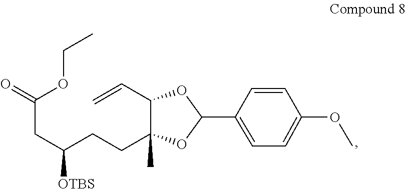

to form Compound 9

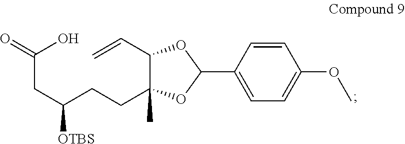

Step 15b) preparing a mixture comprising at least one solvent, a silanyl chloride, and Compound 19rac

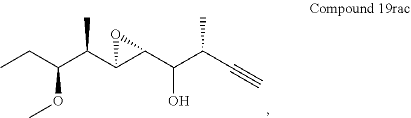 [SMILES: C#C[C@@H](C)C(O)[C@H]1O[C@@H]1[C@H](C)[C@H](CC)OC]

to form a reaction product (e.g., triphenylsilanyl Compound 19rac), and chromatographically purifying the reaction product over silica gel to form Compound 19 or 19R having greater than 99% entantiopurity at carbon 7,

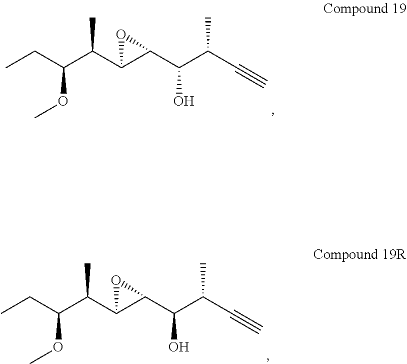

Step 16) preparing a mixture comprising at least one solvent, tributyltin hydride, and Compound 19 or 19R

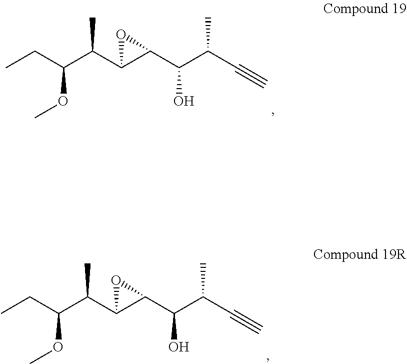

to form Compound 20 or 20R (or instead using trimethyltin hydride to form the corresponding vinyl trimethyl tin compound)

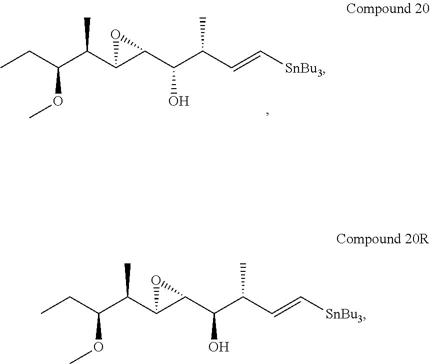

Step 17) preparing a mixture comprising at least one solvent, a Pd catalyst, Compound 20 or 20R (or the corresponding vinyl trimethyl tin compound),
and Compound 14

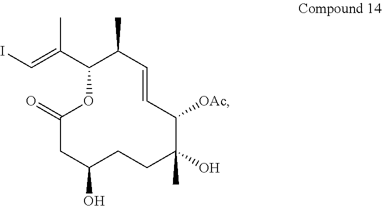 [SMILES: CC(=O)O[C@H]1/C=C/[C@H](C)[C@@H](/C(C)=C/I)OC(=O)C[C@H](O)CC[C@@]1(C)O]

to form Compound A or Compound B

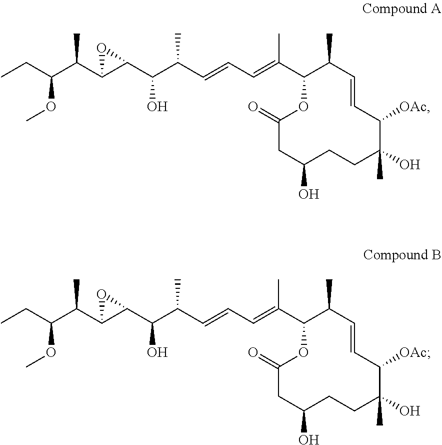 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C ; CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C]

Step 4a) preparing a mixture comprising at least one solvent, Compound 4

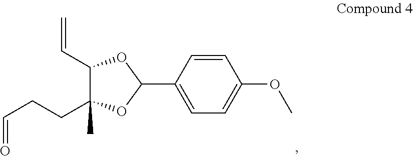

and Compound 21

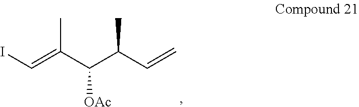

to form Compound 22

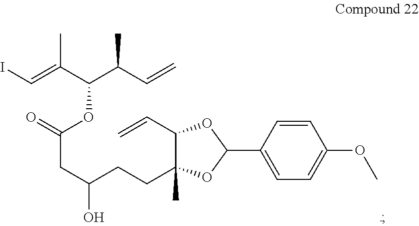

Step 5a) preparing a mixture comprising at least one solvent, and Compound 22

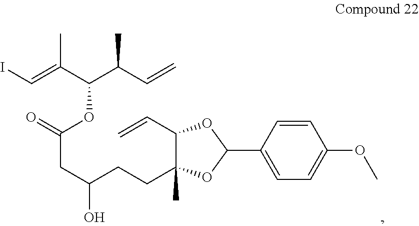

and oxidizing Compound 22 to form Compound 23

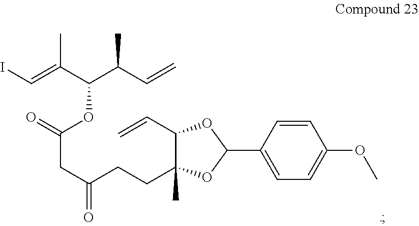

Step 6a) preparing a mixture comprising at least one solvent, and Compound 23

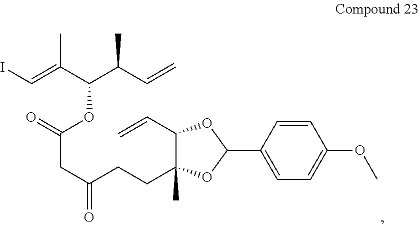

and reducing Compound 23 to form Compound 24

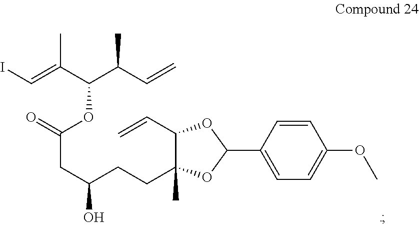

Step 7a) preparing a mixture comprising at least one solvent, tert-butyldimethylsilyl chloride, a base, and Compound 24

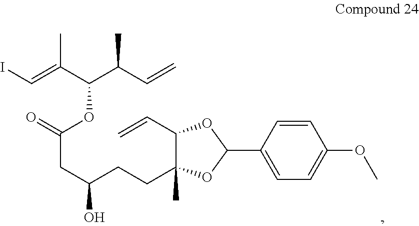

to form Compound 11

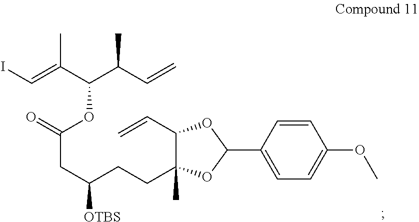

Step 3b) preparing a mixture comprising at least one solvent, and Compound 3

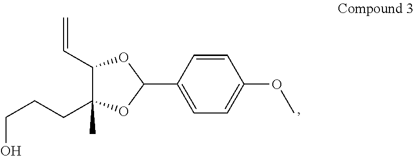

and oxidizing Compound 3 to form Compound 25

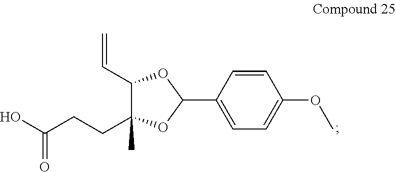

Step 4b) preparing a mixture comprising at least one solvent, a peptide coupling reagent, and Compound 25

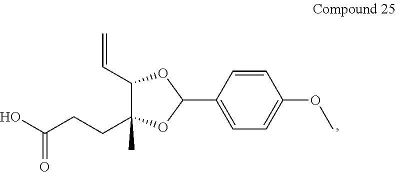

to form Compound 26

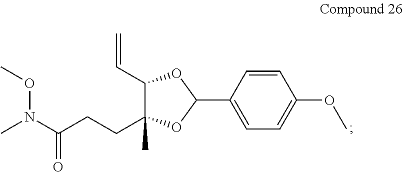

or
Step 5b) preparing a mixture comprising at least one solvent, a dimethylhydroxylamine, a base, and Compound 26

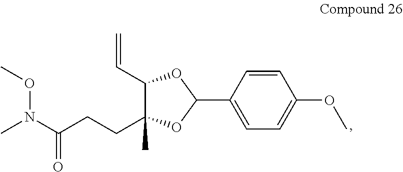

to form Compound 6

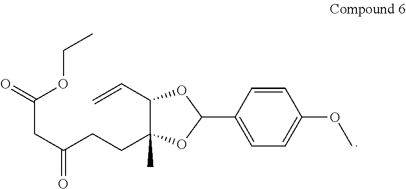

In some embodiments, any of the compounds in the synthetic steps herein can be replaced with the compound's respective generic counterpart provided herein.

In some embodiments regarding Step 6, the enzyme having aldo-keto reductase activity is a KRED or an enzymatically active variant thereof. KREDs require a reduced cofactor NAD (P) H. To reduce costs of commercial synthetic procedures, a cofactor recycling system may be used in conjunction with the enzyme having aldo-keto reductase activity. Thus, in some embodiments, a cofactor regeneration enzyme may be used. In some embodiments, the cofactor regeneration enzyme used herein can include glucose-6-phosphate dehydrogenase, glucose dehydrogenase, and isocitrate dehydrogenase. In some embodiments, the cofactor regeneration enzyme substrate includes a glucose or isocitrate. Step 6 may further include an additional substrate isomerization enzyme, including aconitase, which isomerizes citrate to isocitrate. In some embodiments, the enzymes used in Step 6 are bound to a solid support, are not bound to a solid support, or include a combination of bound and unbound enzymes. In some embodiments, the enzyme includes ketoreductase 1 from Oogatea glycozyma (KRED1-Pglu), alcohol dehydrogenase from Ralstonia sp. (RADH), and alcohol dehydrogenase from Lactobacillus brevis (LbADH). In some embodiments, KREDS include, for example, those classified under the EC numbers of 1.1.1. KREDS may include alcohol dehydrogenase, carbonyl reductase, lactate dehydrogenase, hydroxyacid dehydrogenase, hydroxyisocaproate dehydrogenase, β-hydroxybutyrate dehydrogenase, steroid dehydrogenase, sorbitol dehydrogenase, or aldoreductase, or an enzymatically active variant thereof. NADPH-dependent KREDs include those classified under the EC number of 1.1.1.2. NADH-dependent KREDs include those classified under the EC number of 1.1.1.1. In some embodiments, the KRED enzyme is KRED-A6-P2D5, and the enzymatic reaction takes place in the presence of NADP and a solvent, optionally in the presence of a buffer and a nonionic surfactant having a hydrophilic head and a hydrophobic/lipophilic tail and a hydrophilic-lipophilic balance (HLB) of about 10-20 (e.g., 13 to 15), such as octylphenoxypolyethoxyethanol (IGEPAL CA-630; HLB of 13.4) or a polyethylene glycol p-(1,1,3,3-tetramethylbutyl)-phenyl ether such as Triton-X100 (CAS number 9002-93-1; HLB of 13.4). In some embodiments, the solvent includes isopropyl alcohol, water, or a mixture thereof. Similarly to Step 6, regarding sequential Step 15c/d, an aldo-keto reductase activity may be used to transform Compound 190 to pure Compound 19 or 19R.

In some embodiments, the synthesized compound is a polyketide.

In some embodiments, the polyketide is

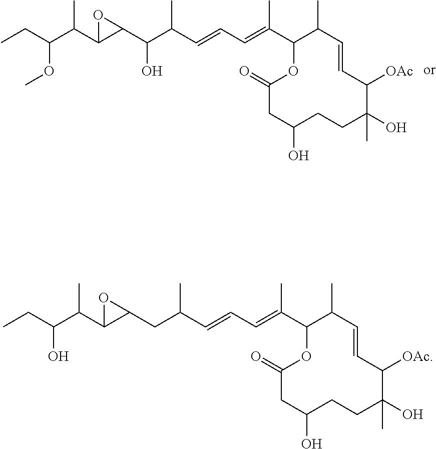 [SMILES: CCC(OC)C(C)C1OC1C(O)C(C)/C=C/C=C(\C)C1OC(=O)CC(O)CCC(C)(O)C(OC(C)=O)/C=C/C1C ; "or" ; CCC(O)C(C)C1OC1CC(C)/C=C/C=C(\C)C1OC(=O)CC(O)CCC(C)(O)C(OC(C)=O)/C=C/C1C]

In some embodiments, the synthesized compound is

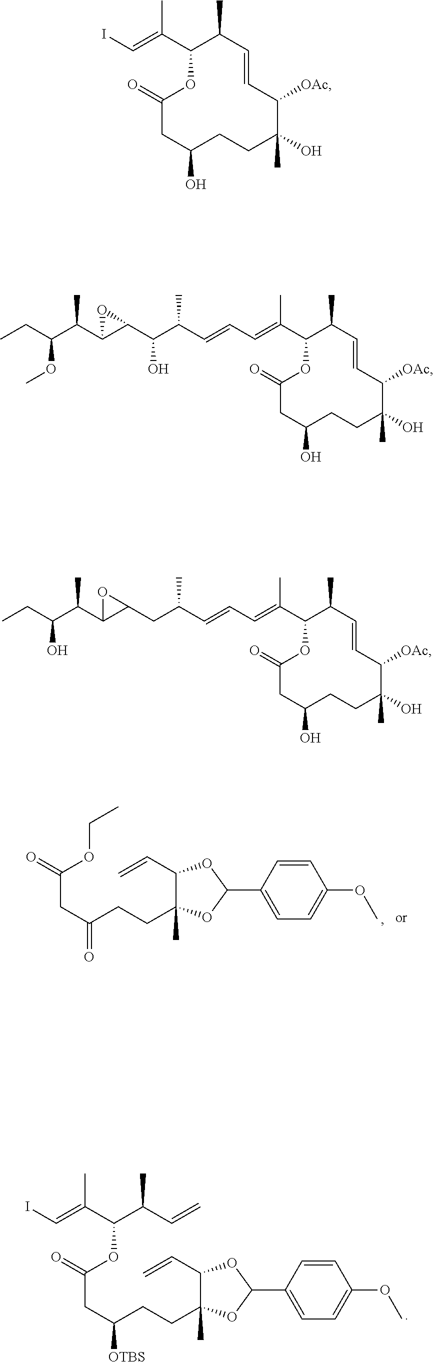

In some embodiments, provided herein are compounds, whether intermediates or polyketides such as Compound A, Compound B, or Pladienolide B, prepared by one or more methods described herein.

In some embodiments, provided herein are compounds, prepared by one or more methods described herein, wherein the compound is:

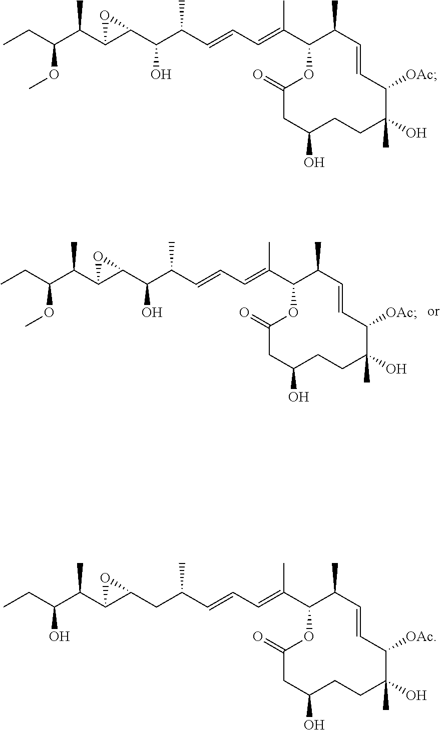 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C ; CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C ; "or" ; CC[C@H](O)[C@@H](C)[C@H]1O[C@@H]1C[C@H](C)/C=C/C=C(\C)[C@H]1OC(=O)C[C@H](O)CC[C@@](C)(O)[C@@H](OC(C)=O)/C=C/[C@@H]1C]

In some embodiments, provided herein are compositions, comprising: 1)

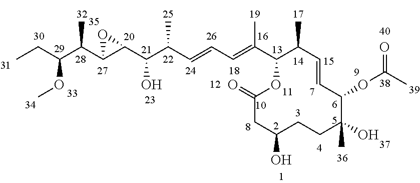 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]

having greater than 99% entantiopurity at carbon 21,

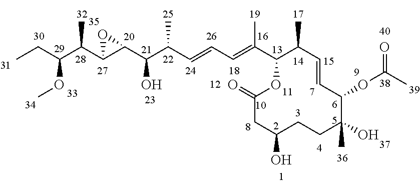 [SMILES: CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C]

having greater than 99% entantiopurity at carbon 21,

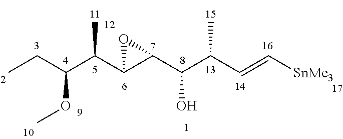

having greater than 99% entantiopurity at carbon 8,

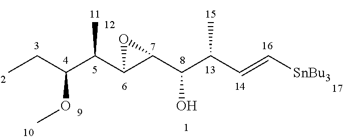

having greater than 99% entantiopurity at carbon 8,

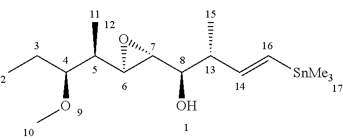

having greater than 99% entantiopurity at carbon 8,

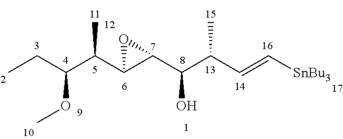

having greater than 99% entantiopurity at carbon 8,

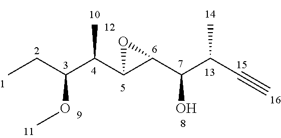 [SMILES: C#C[C@@H](C)[C@@H](O)[C@H]1O[C@@H]1[C@H](C)[C@H](CC)OC]

having greater than 99% entantiopurity at carbon 7, or

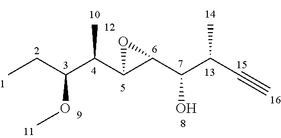 [SMILES: C#C[C@@H](C)[C@H](O)[C@H]1O[C@@H]1[C@H](C)[C@H](CC)OC]

having greater than 99% entantiopurity at carbon 7; and
2) optionally, wherein the composition comprises less than 0.5 ppm Sn (e.g., less than 0.5 ppm Sn other than Sn from the compound above, e.g., the organostannane compound).

In some embodiments, provided herein are compositions, comprising: 1)

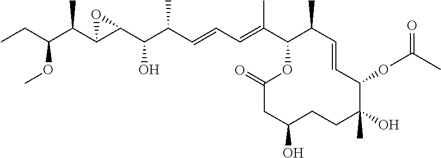 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]

having greater than 99% entantiopurity,

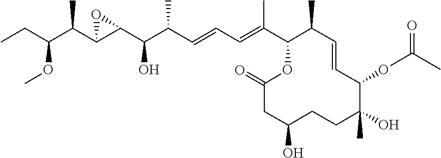 [SMILES: CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C]

having greater than 99% entantiopurity,

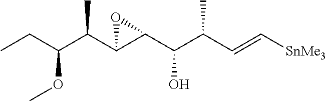

having greater than 99% entantiopurity,

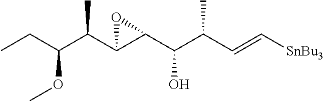

having greater than 99% entantiopurity,

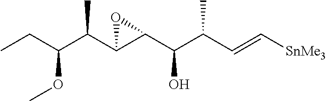

having greater than 99% entantiopurity,

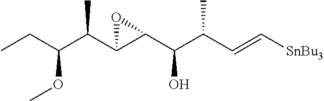

having greater than 99% entantiopurity,

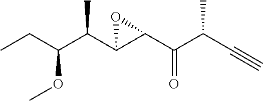 [SMILES: C#C[C@@H](C)C(=O)[C@H]1O[C@@H]1[C@H](C)[C@H](CC)OC]

having greater than 99% entantiopurity,

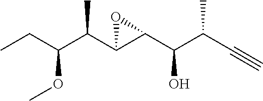 [SMILES: C#C[C@@H](C)[C@@H](O)[C@H]1O[C@@H]1[C@H](C)[C@H](CC)OC]

having greater than 99% entantiopurity, or

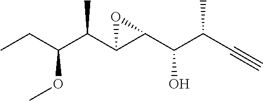 [SMILES: C#C[C@@H](C)[C@H](O)[C@H]1O[C@@H]1[C@H](C)[C@H](CC)OC]

having greater than 99% entantiopurity; and
2) optionally, wherein the composition comprises less than 0.5 ppm Sn (e.g., less than 0.5 ppm Sn other than Sn from the compound above, e.g., the organostannane compound).

Chemical abbreviations used herein include, but are not limited to: CSA for 10-camphorsulfonic acid; DCC for N,N′-dicyclohexylcarbodiimide; DMAP for dimethylaminopyridine; DMSO for dimethyl sulfoxide; GDH for a glutamate dehydrogenase; HPLC for high performance liquid chromatography; IBX for 2-iodoxybenzoic acid; KRED for an aldo-keto reductase; LDA for lithium diisopropylamide; MTBE for methyl tert-butyl ether; NAD or NAD+ for nicotinamide adenine dinucleotide; NADH for the reduced form of NAD+; NADP or NADP+ for nicotinamide adenine dinucleotide phosphate; NADPH for the reduced form of NADP+; ppm for parts per million; TBSCl for tert-butyldimethylsilyl chloride; TBSOTf for tert-butyldimethylsilyl trifluoromethanesulfonate; TEMPO for (2,2,6,6-tetramethylpiperidin-1-yl)oxyl; TFA for trifluoroacetic acid; and THF for tetrahydrofuran.

### Compositions and Uses

As described above, the compounds provided herein, including intermediates and finished compounds, for example Compound A, Compound B, Pladienolide B, and the like, may be prepared as described herein at commercially relevant scales, which renders the compounds further useful in preparative and therapeutic applications.

Thus, in some embodiments, provided herein are compositions comprising a compound provided herein. In some embodiments, the composition is a pharmaceutical composition comprising a compound described herein, such as, but not limited to, Compound A, or the like, prepared by a synthetic method including at least one synthetic step described herein. In some embodiments, the pharmaceutical compositions referred to herein may include at least one pharmaceutically acceptable carrier.

In some embodiments, the compounds described herein, such as, but not limited to, Compound A, or the like, prepared by a synthetic method including at least one synthetic step described herein, are useful in treating a neoplasm. In some embodiments, the neoplasm includes a cancer. In some embodiments, the neoplasm includes a tumor. In some embodiments, the cancer is a malignant cancer. In some embodiments, the cancer is a benign cancer. In some embodiments, the compounds described herein, such as, but not limited to, Compound A, or the like, prepared by a synthetic method including at least one synthetic step described herein, are useful in modulating (e.g., inhibiting) spliceosomal activity. In some embodiments, the compounds described herein, such as, but not limited to, Compound A, or the like, prepared by a synthetic method including at least one synthetic step described herein, are useful in modulating (e.g., inhibiting) ADAR activity. In some embodiments, the compounds described herein, such as, but not limited to, Compound A, or the like, prepared by a synthetic method including at least one synthetic step described herein, are useful in modulating (e.g., inhibiting) RNA-editing activity. Such activity may occur in vivo or in vitro, including within a subject, such as a human subject.

In some embodiments, provided herein are methods of treating a neoplasm in a subject in need thereof, comprising administering a therapeutically effective amount of an active agent prepared as described herein, or a composition thereof, to the subject.

In some embodiments, provided herein are methods of modulating spliceosomal activity in a cell, comprising contacting the cell with an effective amount of an active agent prepared as described herein, or a composition thereof.

In some embodiments, provided herein are methods of modulating adenosine deaminase acting on RNA (ADAR) activity in a cell, comprising contacting the cell with an effective amount of an active agent prepared as described herein, or a composition thereof.

In some embodiments, the cell is in vitro. In some embodiments, the cell is in vivo, e.g., in a subject, e.g., in a mammalian subject, e.g., a human subject.

Accordingly, actual dosage levels of the active ingredients (e.g. Compound A, Compound B, Pladienolide B, and the like) prepared as described herein, the compositions, or the pharmaceutical compositions provided herein may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health, and prior medical history of the patient being treated, and like factors well-known in the medical arts. A medical doctor, e.g., physician or veterinarian, having ordinary skill in the art may readily determine and prescribe the effective amount of the pharmaceutical composition required. For example, the physician or veterinarian could start doses of the compounds employed in the pharmaceutical composition at levels lower than that required in order to achieve the desired therapeutic effect and gradually increase the dosage until the desired effect is achieved.

Routes of administration of include, without limitation, oral, nasal, rectal, intravaginal, parenteral, buccal, sublingual, or topical. In some embodiments, the oral or nasal route of administration is an oral inhalational or nasal inhalational route of administration. The compounds for use as described herein may be formulated for administration by any suitable route to achieve the particular method being applied.

In some embodiments, provided herein are packaged compounds, packaged compositions, or packaged pharmaceutical compositions, comprising a container holding a therapeutically effective amount of a compound described herein, such as, but not limited to, Compound A, or the like, prepared by a synthetic method described herein, and instructions for using the compound in accordance with one or more of the methods provided herein.

The present compounds and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g. Irgacure 184, 2959), preferably water-soluble initiators (e.g. Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g. by the addition of specific product information leaflets) into suitable containers (boxes, etc.).

According to further embodiments, the present compounds can also be provided in kit form combined with other components necessary for administration of the material to the patient. For example, disclosed kits, such as for use in the treatment of cancer, can further comprise, for example, administration materials.

The kits are designed in various forms based on the specific deficiencies they are designed to treat.

The compounds or compositions provided herein may be prepared and placed in a container for storage at ambient or elevated temperature. When the compound or composition is stored in a polyolefin plastic container as compared to a polyvinyl chloride plastic container, discoloration of the compound or composition, or sorption of the compound with the surface of the container, may be reduced, whether dissolved or suspended in a liquid composition (e.g., an aqueous or organic liquid solution), or as a solid. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce exposure of the container's contents to infrared light, or also include a second component with such a capacity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper, cardboard, paperboard, metallic film, or foil, or a combination thereof, container to further reduce exposure of the container's contents to UV, visible, or infrared light. The compounds or compositions provided herein may need storage lasting up to, or longer than, three months; in some cases up to, or longer than one year. The containers may be in any form suitable to contain the contents; for example, a bag, a bottle, or a box.

The following examples further illustrate aspects of the present disclosure. However, they are in no way a limitation of the teachings or disclosure as described herein.

### EXAMPLES

### Example 1: Synthesis of Intermediates and Compound 14 as Shown in FIG. 1

Methods and materials are compared against Chan 2020 and WO 2021/026273 A1.
Step 1:10-Camphorsulfonic acid (CSA), MeOH, at 50° C. for 12 h, then buffer with Ambersep 900 (OH).

Advantage: Previous methods of synthesis require large volumes of solvent to convert Compound 1 to Compound 3. The present method enables scalable preparation with 8 volumes of solvent, a key step in enabling access to kilogram production of Compound 3.

Exemplary Procedure: To a solution of Compound 1 (10 g, 27.58 mmol, 1 eq.) in MeOH (80 mL) was added CSA (961.03 mg, 4.14 mmol, 0.15 eq.), the mixture was stirred at 50° C. for 12 h. Ambersep 900 (OH) (5 g) (a strongly basic anion exchange resin; includes resin-bound hydroxide via quaternary ammonium) was added to adjust the pH between 7-8, the solution was filter and concentrated. The crude (91% by NMR) was used directly in the next step.
Step 2: anisaldehyde dimethyl acetal, Amberlyst 15 (H), CH₂Cl₂, then buffer with Ambersep 900 (OH).

Advantage: As above in Step 1.

Exemplary Procedure: 1-(Dimethoxymethyl)-4-methoxy-benzene (10.05 g, 55.18 mmol, 9.40 mL, 2 eq.) and Amberlyst 15 (H) (1 g, 27.59 mmol) (a strongly acidic cation exchange resin; includes resin-bound sulfonic acid) was added sequentially to the crude product from Step 1 (4.42 g, 27.59 mmol, 1 eq.) in CH₂Cl₂ (120 mL) at 20° C. The mixture was stirred at 20° C. for 2 h. The reaction mixture was filtered to remove acid resin. Ambersep 900 (OH) (5 g) was added to adjust the pH 7-8, filtered and concentrated under reduced pressure to give a residue. Purification by column chromatography using a gradient of heptanes to 3:1 heptane/EtOAc afforded pure 2 (4.7 g, 61%).
Step 3: KBr, TEMPO, NaHCO₃, NaClO, CH₂Cl₂.

Advantage: Addition of KBr as a co-catalyst.

Exemplary Procedure: As otherwise described in Chan 2020 or WO 2021/026273 A1.
Step 4: EtOAc, LDA, THF.

Advantage: High yielding addition to the aldehyde.

Exemplary Procedure: This procedure was conducted under N₂ atmosphere until the aqueous quench. LDA (0.69 M, 158 mL, 1.50 eq.) was added slowly to a solution of EtOAc (8.29 g, 94.1 mmol, 9.21 mL, 1.30 eq.) in THF (180 mL) at −70° C. The mixture was stirred at −70° C. for 1 h. A solution of Compound 4 (20.0 g, 72.4 mmol, 1.00 eq.) in THF (60 mL) was added at −60° C. The mixture was stirred at −70° C. for 0.5 h. The reaction mixture was quenched with saturated NH₄Cl (100 mL) at −40° C. The resulting solution was extracted with EtOAc (2×70 mL). The combined organic layers were washed with H₂O and dried over Na₂SO₄. After filtration via filter paper, the organic layer was concentrated under reduced pressure to give a residue. Purification by column chromatography using a gradient of petroleum ether to 15:1 petroleum ether/EtOAc afforded pure Compound 5 (23.0 g, 84.9%).
Step 5: Two methods were found viable: IBX (3 eq.), EtOAc, 70° C., 24 h, or DCC (3 eq.), pyridine·TFA (0.5 eq.), DMSO, 20° C., 12 h, 60% (Pfitzner-Moffatt).

Advantage: Enables oxidation to an achiral material enabling chiral reduction in Step 6, the IBX step has advantages on the gram scale while the Pfitzner-Moffatt can be used to reach kg scales.

Exemplary Procedure (IBX): IBX (22.5 g, 80.3 mmol) was added to Compound 5 (9.8 g, 26.7 mmol) in EtOAc (300 mL). The mixture was heated with rapid stirring for 24 h at 70° C. After this period, it was cooled to room temperature and filtered. Pure Compound 6 (5.9 g, 61%) was obtained by flash plug chromatography eluting with aliquots of 4:1 heptanes: EtOAc, 3:1 heptanes: EtOAc, and 2:1 heptanes: EtOAc.

Exemplary Procedure (Pfitzner-Moffatt): Alcohol Compound 5 (50 mg, 0.14 mmol) was dissolved in DMSO (5 mL) and DCC (84.9 mg, 0.41 mmol) was added. After stirring at room temperature for 12 h, H₂O (20 mL) was added. The crude product was obtained by extraction with EtOAc (4×50 mL), drying with Na₂SO₄, and concentration by rotary evaporation. Pure Compound 6 (0.30 g, 60%) was obtained by flash plug chromatography eluting with aliquots of 4:1 heptanes: EtOAc, 3:1 heptanes: EtOAc, and 2:1 heptanes: EtOAc.
Step 6: GDH, ketoreductase, D-glycose, phosphate buffer pH7.

Advantage: A highly stereoselective reduction process with chiral purity of 99.6%.

Exemplary Procedure: To a mixture of D-glucose (10.8 g, 59.9 mmol, 6.04 eq.), GDH (360 mg) from Bacillus megaterium, NADP+ (360 mg), and KRED (3.60 g) from Hansenula polymorpha in phosphate buffer pH about 6.5-7 (180 mL) was added Compound 6 (3.60 g, 9.93 mmol, 1.00 eq.) in DMSO (10 mL) drop wise at 30° C., and the mixture was stirred at 30° C. for 18 h. HPLC analysis showed 15.6% Compound 6 (retention time of 3.033 min) remained and 75.4% product Compound 7 (retention time of 2.813 min) was detected. The reaction mixture was quenched by CH₃CN (320 mL), filtered and the filtrate collected. The resulting solution was diluted with EtOAc (150 mL), the organic phase separated, washed with brine (100 mL), dried over Na₂SO₄, filtered and concentrated. Compound 7 (3.50 g, crude, chiral purity: 99.6%) was obtained as yellow oil.
Step 7: TBSCl, imidazole, CH₂Cl₂ or TBSOTf, 2,6-lutidine, CH₂Cl₂.

Advantage: TBSCl procedure is more scalable than the TBSOTf procedure.

Exemplary Procedure (TBSCl): Imidazole (2.00 g, 29.4 mmol, 3.06 eq.) and TBSCl (3.00 g, 19.90 mmol, 2.44 mL, 2.07 eq.) were added sequentially to a solution of Compound 7 (3.50 g, 9.60 mmol, 1.00 eq.) in CH₂Cl₂ (3 mL) at 25° C. The mixture was stirred at 25° C. for 12 h. H₂O (30 mL) was added to the reaction mixture and the resulting solution was extracted with CH₂Cl₂ (2×20 mL), the combined organic layers were washed with H₂O and dried over Na₂SO₄. After filtration via filter paper, the organic layer was concentrated under reduced pressure to give a residue. Purification by column chromatography using a gradient of petroleum ether to 10:1 petroleum ether/EtOAc afforded pure Compound 8 (4 g, crude).
Step 8: KOH, MeOH.

Advantage: Provides facile conversion to Compound 9.

Exemplary Procedure: To a solution of Compound 8 (4.00 g, 8.36 mmol, 1.00 eq.) in H₂O (10 mL) and MeOH (40 mL) was added KOH (1.20 g, 21.4 mmol, 2.56 eq.) at 20° C., then the mixture was stirred at 20° C. for 1 h. Under 0° C., the reaction mixture was adjusted to pH 5-6 with 5% citric acid (150 mL), extracted with CH₂Cl₂ (200 mL), washed with H₂O (150 mL), dried over Na₂SO₄, filtered and the filtrate was concentrated under reduced pressure to give a residue. Purification by column chromatography using a gradient of petroleum ether to 20:1 petroleum ether/EtOAc afforded pure Compound 9 (2.5 g, 66.4%).
Step 9: Exemplary Procedure: As described in Chan 2020 or WO 2021/026273 A1.
Step 10: Exemplary Procedure: As described in Chan 2020 or WO 2021/026273 A1.
Step 11: Exemplary Procedure: As described in Chan 2020 or WO 2021/026273 A1.
Step 12: Exemplary Procedure: As described in Chan 2020 or WO 2021/026273 A1.

### Example 2: Synthesis of intermediates and Compound A as shown in FIG. 2

Methods and materials are compared against Chan 2020 and WO 2021/026273 A1.
Step 13: Exemplary Procedure: As described in Chan 2020 or WO 2021/026273 A1.
Step 14: Exemplary Procedure: As described in Chan 2020 or WO 2021/026273 A1.
Step 15: Exemplary Procedure: Synthesis as described in Chan 2020 or WO 2021/026273 A1, but with improved method of purification (Step 15b and/or Step 15c/d).

Advantage: Chromatographic and chemoenzymatic methods have been developed to ensure that Compound 19 can be made with >99% enantiopurity and <1 ppm of organostannane byproducts.
Step 15b: Conversion of a mixture of Compounds 19 and 19R to pure Compound 19 or 19R.

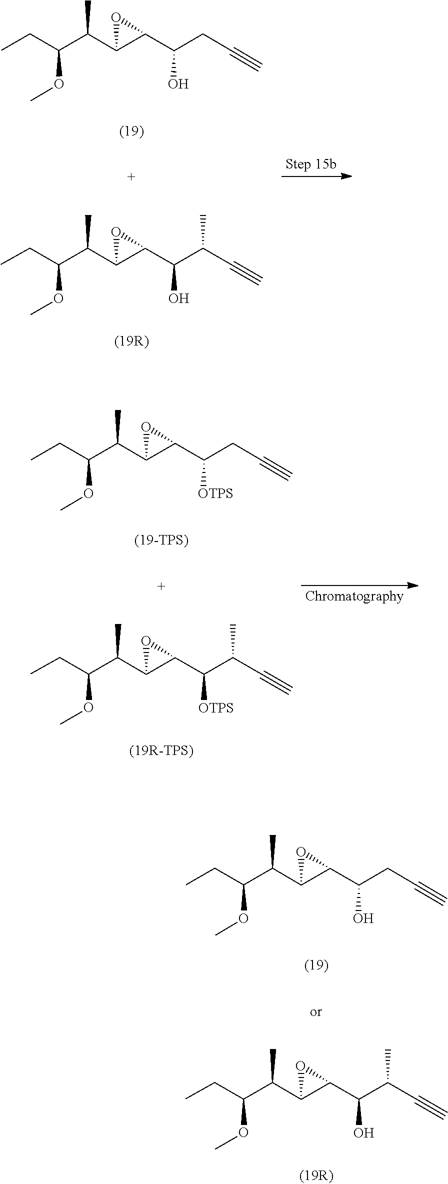

Chlorotriphenylsilane (3.91 g, 13.26 mmol, 0.3 eq.) was added to a solution of crude Compound 19 (10 g, 44.19 mmol, 1 eq.; a mixture of Compound 19 and Compound 19R) and DMAP (539.82 mg, 4.42 mmol, 0.1 eq.) and pyridine (17.48 g, 220.93 mmol, 17.83 mL, 5 eq.) in CH₂Cl₂ (70 mL) at 25° C. The mixture was stirred at 25° C. for 12 h. TLC (5:1 petroleum ether/EtOAc) indicated one new spot formed. The reaction mixture was quenched by addition of H₂O (150 mL) at 0° C., and then extracted with MTBE (2×150 mL). The combined organic layers were washed with 10% citric acid until the pH of aqueous phase was 3-4. Then the organic layer was dried over Na₂SO₄, filtered and concentrated under reduced pressure to give a residue (22.5 g). The residue was dissolved in MTBE (150 mL) and 34 g of silica gel (100-200 mesh) was added. Then it was concentrated under reduced pressure to remove the solvent to get a residue. Then 170 g of silica gel (200-300 mesh) was packed into a chromatographic column. The residue was purified by column chromatography (170 g of silica gel 200-300 mesh, 100:0 to 1:1 petroleum ether/EtOAc). Compound 19R (eluted with 50:1 petroleum either/EtOAc) and Compound 19 (eluted with 3:1 petroleum ether/EtOAc) were collected respectively, then concentrated to give 5.5 g of enantiopure Compound 19.
Step 15c/d: Conversion of a mixture of Compounds 19 and 19R to pure Compound 19.

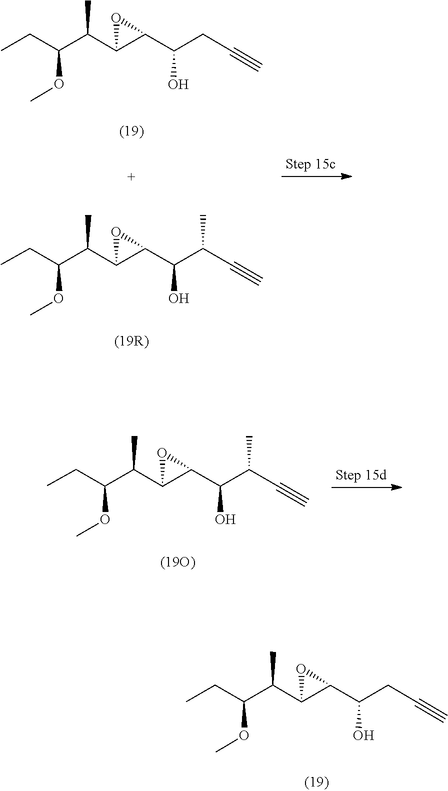 [SMILES: C#CC[C@H](O)[C@H]1O[C@@H]1[C@H](C)[C@H](CC)OC ; C#C[C@@H](C)[C@@H](O)[C@H]1O[C@@H]1[C@H](C)[C@H](CC)OC ; C#C[C@@H](C)[C@@H](O)[C@H]1O[C@@H]1[C@H](C)[C@H](CC)OC ; C#CC[C@H](O)[C@H]1O[C@@H]1[C@H](C)[C@H](CC)OC]

Exemplary Procedure: 2-Iodoxybenzoic acid (IBX) (8.4 g, 30 mmol) was added to a solution of crude Compound 19 (5.01 g, 22.1 mmol; a mixture of Compound 19 and Compound 19R) in a 1 L flask in anhydrous EtOAc (500 mL). The mixture was stirred for 4 h at 23° C., at which point the resulting mixture was, filtered through a pad of silica gel (200 g) and concentrated to yield crude colorless oil containing Compound 190. This material was subjected to an enzymatic reduction. This began by dissolving the colorless oil containing Compound 190 in isopropyl alcohol (200 mL). This was added to buffer (3 L) containing Triton X100 (60 mL). Enzyme KRED-A6-P2D5 (0.2 g) and NADP (0.05 g). The reaction as stirred at 42° C. for 28 h. Compound 19 was obtained by extraction three times with EtOAc (500 mL) washing with brine (200 mL). The organic phases were combined, dried with Na₂SO₄ and concentrated on a rotary evaporator. Pure Compound 19 (3.8 g, 75%) was obtained as a colorless oil by flash chromatography, eluting with a gradient of hexanes to 1:3 Et2O/hexanes.
Step 16: Conversion of Compound 19 to vinylstannane 20.

Exemplary Procedure: PdCl₂ (PPh₃)₂ (1.55 g, 2.21 mmol) was added to a solution of Compound 19 (5.01 g, 22.1 mmol) in a 500 mL flask in anhydrous THF (200 mL). The mixture was cooled to 0° C. and n-BusSnH (17.9 mL, 66.3 mmol) was added dropwise. The mixture was stirred for 45 min at 0° C., at which point the resulting mixture was concentrated to yield a black crude oil. The material was extracted into hexanes, filtered through a pad of Celite and was eluted with hexanes. The elutant was concentrated on a rotary evaporator, and this process was repeated twice until a clear black solution was achieved.

Pure Compound 20 (5.72 g, 50%) was obtained as a mixture of 1:5 a: β regioisomers by flash chromatography, eluting with a gradient of hexanes to CH₂Cl₂ to 1:20 Et2O/CH₂Cl₂. The desired regioisomer can be obtained in 95+% purity by additional flash chromatography, eluting with a gradient of hexanes to CH₂Cl₂ to 1:20 Et₂O/CH₂Cl₂.

A similar procedure is performed with Me₃SnH instead of n-BusSnH to prepare the corresponding trimethyl vinylstannane compound.
Step 17: Combination of Compound 20 and Compound 14 to form Compound A by a Stille coupling.

Exemplary Procedure: Compound 20 (1.33 g, 2.57 mmol) and Compound 14 (1.00 g, 2.14 mmol) were combined in a 100 mL flask and dried via rotary evaporation of benzene. To the mixture was then sequentially added CuCl (0.425 g, 4.29 mmol), KF (0.249 g, 4.29 mmol) and XPhos Pd G2 (0.169 g, 0.214 mmol) and anhydrous t-BuOH (25 mL). The reaction vessel was purged under Ar, heated to 50° C. and stirred overnight, at which point solution turns into a gray cloudy mixture. The mixture was then filtered through a plug of Celite and eluted with acetone (200 mL). The elutants were concentrated on a rotary evaporator to yield a crude brown wax. Pure Compound A (1.21 g, 80%) was obtained as a light yellow wax by flash chromatography over neutral silica gel, eluting with a gradient of hexanes to 1:3 acetone/hexanes.

A similar procedure is performed with corresponding trimethyl vinylstannane compound instead of Compound 20 to prepare Compound A.

### Example 3: Synthesis of Intermediates and Compound B Similar to that Shown in FIG. 2 for Compound A

Compound 19R and Compound 20R are prepared as described in Example 2.
Step 18: Combination of Compound 20R and Compound 14 to form Compound B by a Stille coupling.

Exemplary Procedure: Compound 20R (1.33 g, 2.57 mmol) and Compound 14 (1.00 g, 2.14 mmol) are combined in a 100 mL flask and dried via rotary evaporation of benzene. To the mixture is then sequentially added CuCl (0.425 g, 4.29 mmol), KF (0.249 g, 4.29 mmol) and XPhos Pd G2 (0.169 g, 0.214 mmol) and anhydrous t-BuOH (25 mL). The reaction vessel is purged under Ar, heated to 50° C. and stirred overnight, at which point solution turns into a gray cloudy mixture. The mixture is then filtered through a plug of Celite and eluted with acetone (200 mL). The elutants are concentrated on a rotary evaporator to yield a crude concentrate. Pure Compound B (1.21 g, 80%) is obtained by flash chromatography over neutral silica gel, eluting with a gradient of hexanes to 1:3 acetone/hexanes.

A similar procedure is performed with corresponding trimethyl vinylstannane compound instead of Compound 20R to prepare Compound B.

We claim:

1. A compound, selected from: 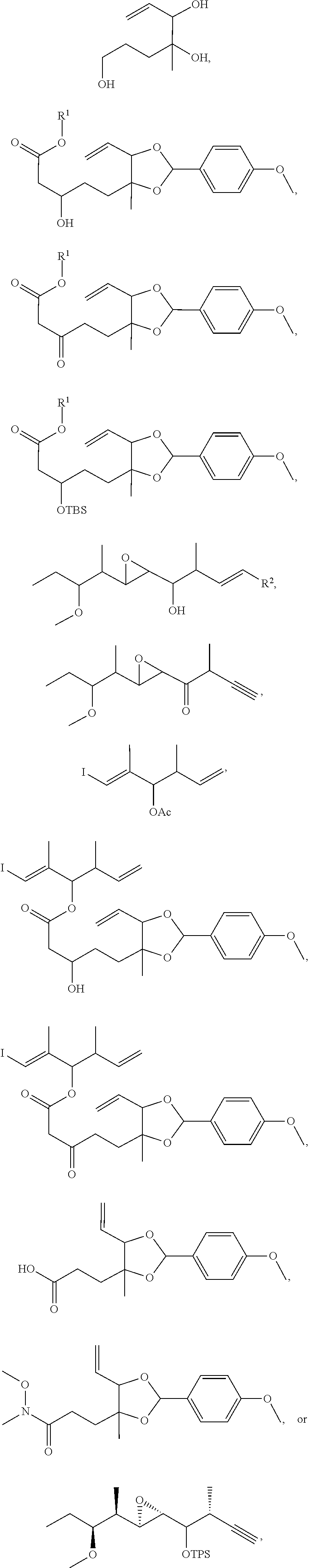
or a salt thereof,
wherein
R¹ is C₁₋₆ alkyl, and
R² is an organotin moiety including a Tin (Sn) atom covalently linked with the carbon to which R² is attached.

2. The compound of claim 1, selected from: 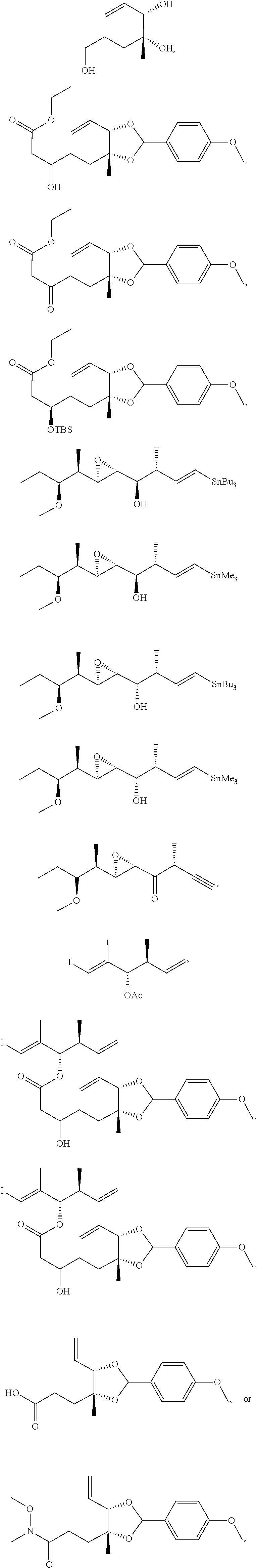
or a salt thereof.

3. The compound of claim 1, selected from:

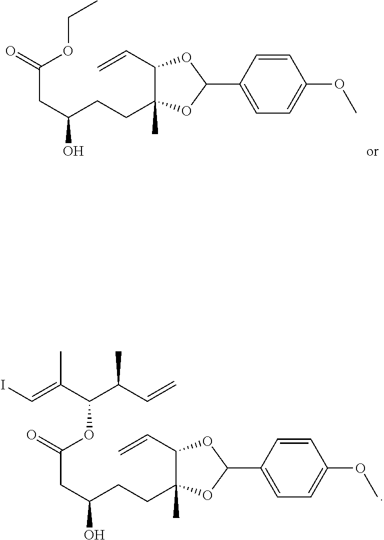

4. A composition, comprising the compound of one of claims 1-3.

5. The compound or the composition of one of claims 1-4, for use in the preparation of a synthesized compound.

6. A method of preparing a synthesized compound, comprising contacting the compound of claim 1 with one or more reagents to form the synthesized compound.

7. A method of preparing the compound of claim 1, comprising contacting a precursor compound with one or more reagents to form the compound of claim 1.

8. A method of preparing a synthesized compound, comprising at least one of the following steps:
Step 1) preparing a mixture comprising at least one solvent, 10-camphorsulfonic acid, and Compound 1 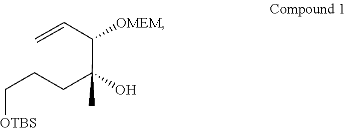
to form Compound 2 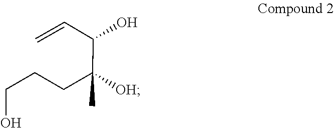
Step 2) preparing a mixture comprising at least one solvent, an acid, 1-(dimethoxymethyl)-4-methoxy-benzene, and Compound 2 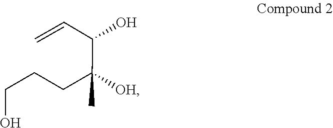
to form Compound 3 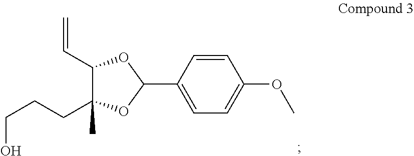
Step 4) preparing a mixture comprising at least one solvent, lithium diisopropylamide, and Compound 4 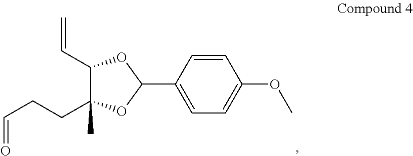
to form Compound 5 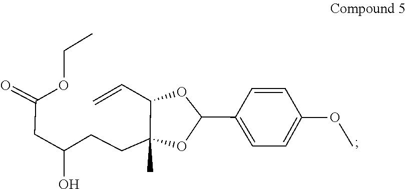
Step 5) preparing a mixture comprising at least one solvent, 2-iodoxybenzoic acid, and Compound 5 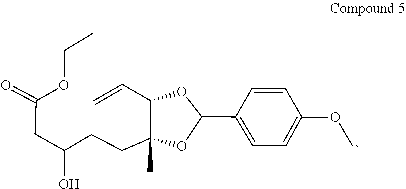
to form Compound 6 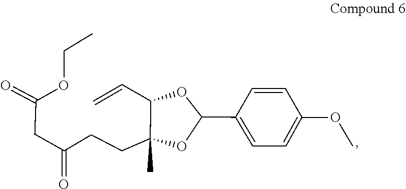
or
preparing a mixture comprising at least one solvent, N,N′-dicyclohexylcarbodiimide, pyridine·trifluoroacetic acid, and Compound 5 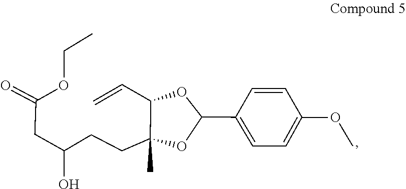
to form Compound 6 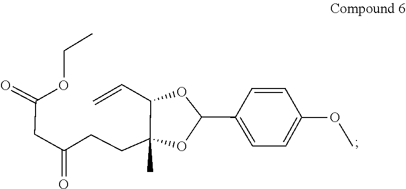
Step 6) preparing a mixture comprising at least one solvent, an enzyme having aldo-keto reductase activity, a reducing cofactor selected from NADPH or NADH, an auxiliary enzyme and substrate for cofactor regeneration, and Compound 6 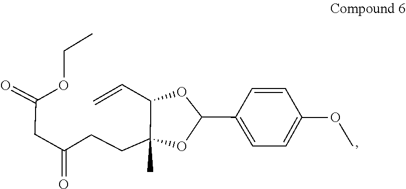
to form Compound 7 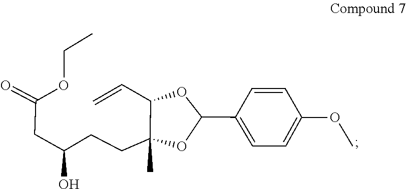
Step 7) preparing a mixture comprising at least one solvent, tert-butyldimethylsilyl chloride, an imidazole, tert-butyldimethylsilyl trifluoromethanesulfonate, a base, and Compound 7 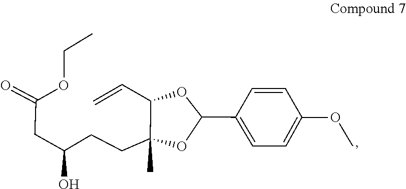
to form Compound 8 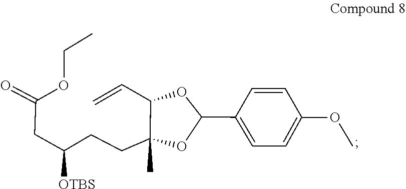
Step 8) preparing a mixture comprising at least one solvent, a base, and Compound 8 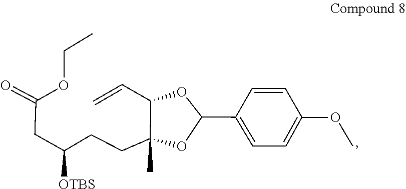
to form Compound 9 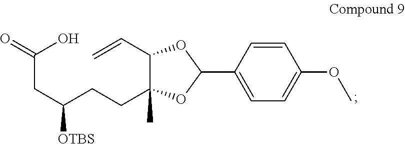
Step 15b) preparing a mixture comprising at least one solvent, a silanyl chloride, Compound 19, and Compound 19 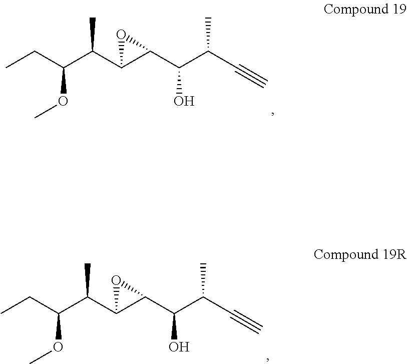
to form a reaction product, and chromatographically purifying the reaction product over silica gel to form Compound 19 or Compound 19R having an EE of at least 99%;
Step 15c) preparing a mixture comprising at least one solvent, Compound 19, Compound 19R, and an oxidizing agent 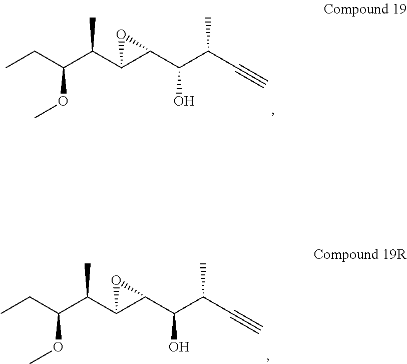
to form Compound 19O; 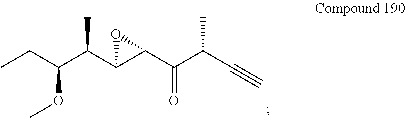 [SMILES: C#C[C@@H](C)C(=O)[C@H]1O[C@@H]1[C@H](C)[C@H](CC)OC]
Step 15d) preparing a mixture comprising at least one solvent, an enzyme having aldo-keto reductase activity, a reducing cofactor selected from NADPH or NADH, an auxiliary enzyme and substrate for cofactor regeneration, and Compound 190 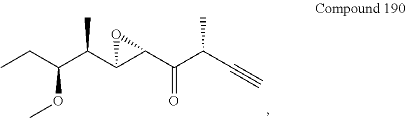 [SMILES: C#C[C@@H](C)C(=O)[C@H]1O[C@@H]1[C@H](C)[C@H](CC)OC]
to form Compound 19 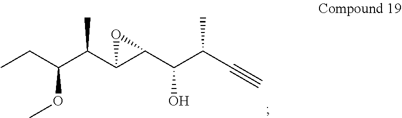 [SMILES: C#C[C@@H](C)[C@H](O)[C@H]1O[C@@H]1[C@H](C)[C@H](CC)OC]
Step 16) preparing a mixture comprising at least one solvent, tributyltin hydride, and Compound 19 or Compound 19R 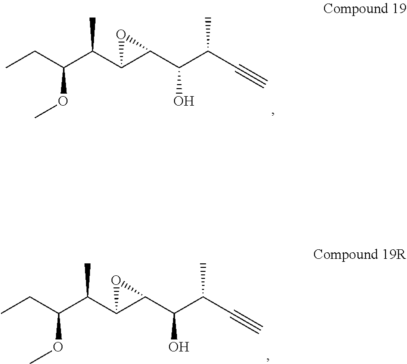
to form Compound 20 or Compound 20R (or instead using trimethyltin hydride to form the corresponding vinyl trimethyl tin compound) 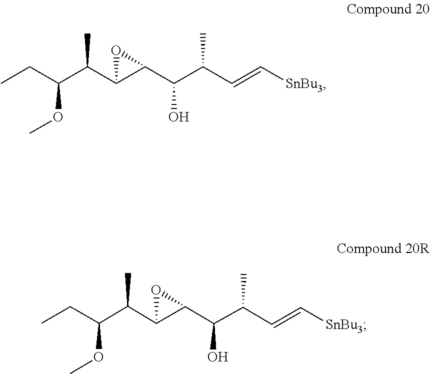
Step 17) preparing a mixture comprising Compound 20 or Compound 20R, and Compound 14 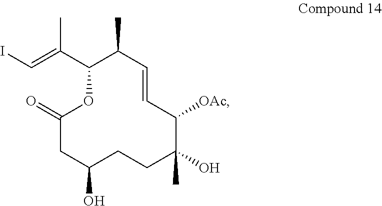 [SMILES: CC(=O)O[C@H]1/C=C/[C@H](C)[C@@H](/C(C)=C/I)OC(=O)C[C@H](O)CC[C@@]1(C)O]
to form Compound A or Compound B 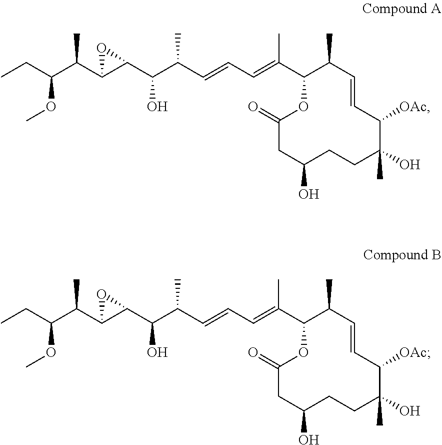 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C ; CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C]
Step 4a) preparing a mixture comprising at least one solvent, Compound 4 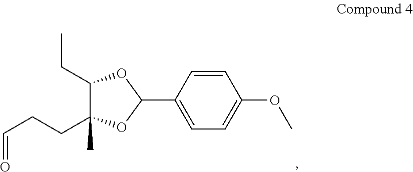
and Compound 21 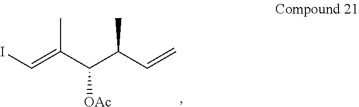
to form Compound 22 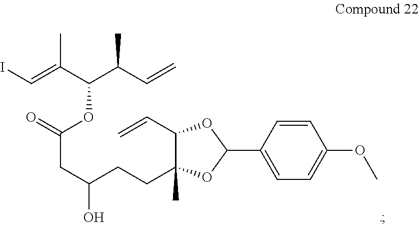
Step 5a) preparing a mixture comprising at least one solvent, and Compound 22 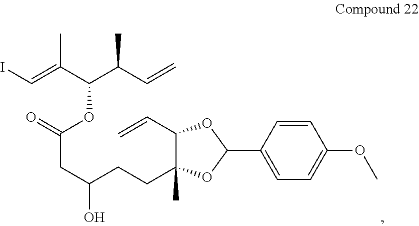
and oxidizing Compound 22 to form Compound 23 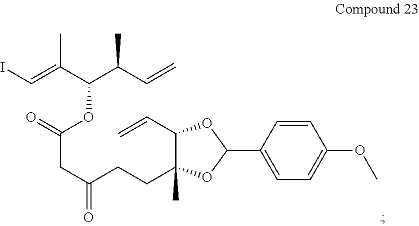
Step 6a) preparing a mixture comprising at least one solvent, and Compound 23 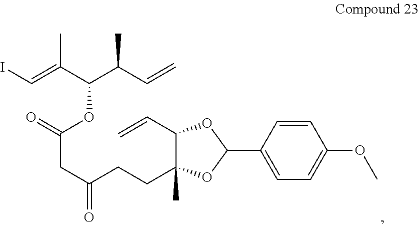
and reducing Compound 23 to form Compound 24 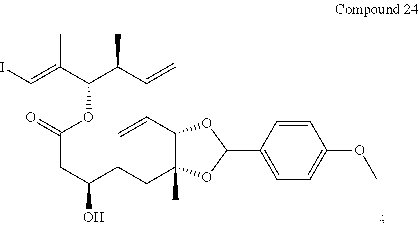
Step 7a) preparing a mixture comprising at least one solvent, tert-butyldimethylsilyl chloride, a base, and Compound 24 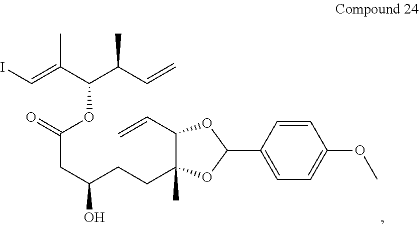
to form Compound 11 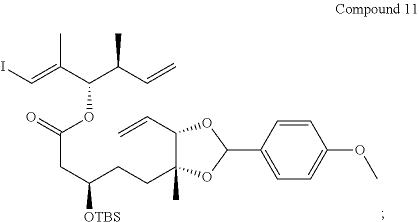
Step 3b) preparing a mixture comprising at least one solvent, and Compound 3 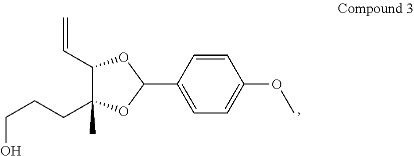
and oxidizing Compound 3 to form Compound 25 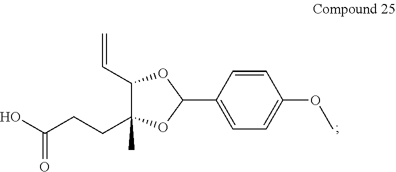
Step 4b) preparing a mixture comprising at least one solvent, a peptide coupling reagent, and Compound 25 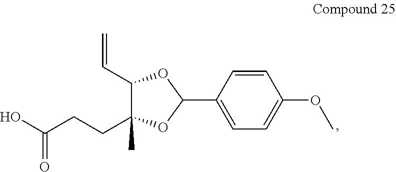
to form Compound 26 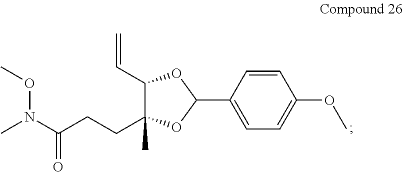
or
Step 5b) preparing a mixture comprising at least one solvent, a dimethylhydroxylamine, a base, and Compound 26 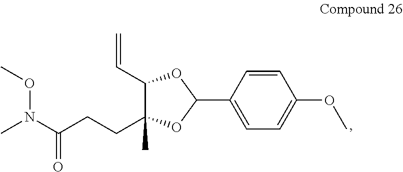
to form Compound 6

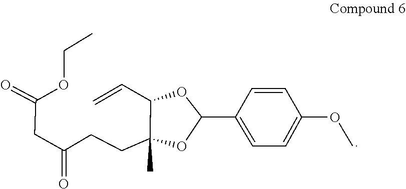

9. The method of claim 8, wherein the synthesized compound is a polyketide.

10. The compound of claim 9, wherein the polyketide is

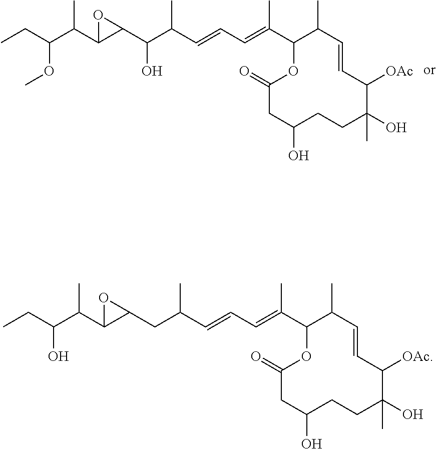 [SMILES: CCC(OC)C(C)C1OC1C(O)C(C)/C=C/C=C(\C)C1OC(=O)CC(O)CCC(C)(O)C(OC(C)=O)/C=C/C1C ; "or" ; CCC(O)C(C)C1OC1CC(C)/C=C/C=C(\C)C1OC(=O)CC(O)CCC(C)(O)C(OC(C)=O)/C=C/C1C]

11. The method of claim 8, wherein the synthesized compound is

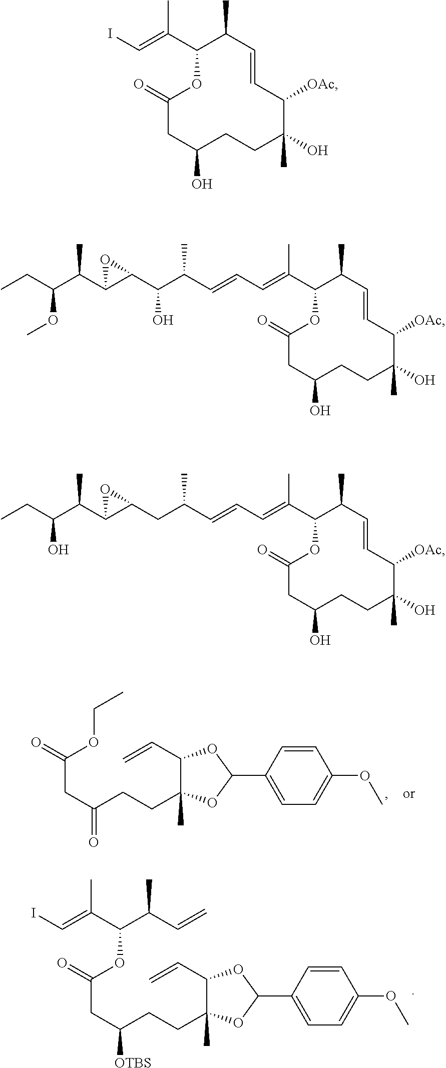

12. A compound, prepared by the method of claim 8, wherein the compound is:

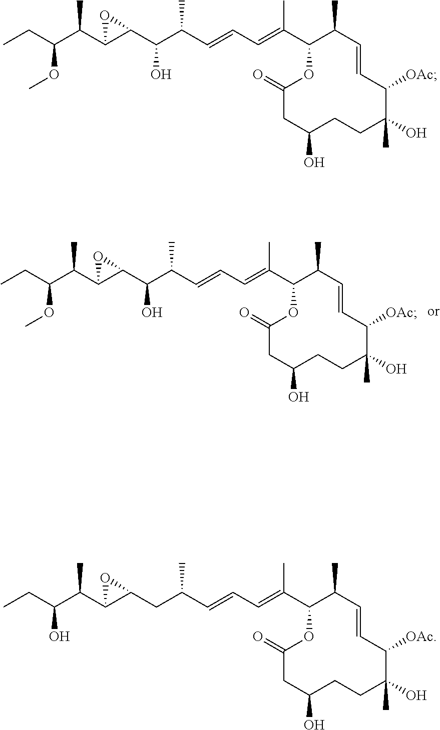 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C ; CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C ; "or" ; CC[C@H](O)[C@@H](C)[C@H]1O[C@@H]1C[C@H](C)/C=C/C=C(\C)[C@H]1OC(=O)C[C@H](O)CC[C@@](C)(O)[C@@H](OC(C)=O)/C=C/[C@@H]1C]

13. A composition, comprising:
1) a compound of a formula selected from: 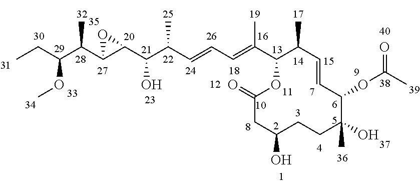 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]
the composition having greater than 99% entantiopurity at carbon 21 of the formula, 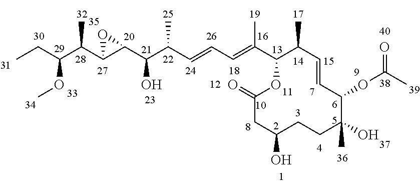 [SMILES: CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C]
the composition having greater than 99% entantiopurity at carbon 21 of the formula, 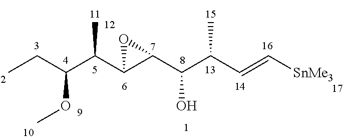
the composition having greater than 99% entantiopurity at carbon 8 of the formula, 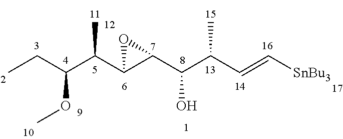
the composition having greater than 99% entantiopurity at carbon 8 of the formula, 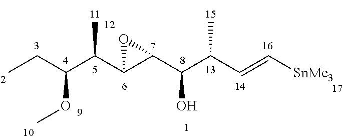
the composition having greater than 99% entantiopurity at carbon 8 of the formula, 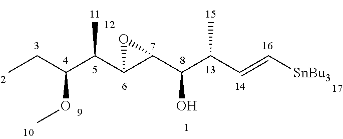
the composition having greater than 99% entantiopurity at carbon 8 of the formula, 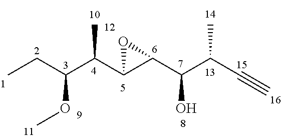 [SMILES: C#C[C@@H](C)[C@@H](O)[C@H]1O[C@@H]1[C@H](C)[C@H](CC)OC]
the composition having greater than 99% entantiopurity at carbon 7 of the formula, or 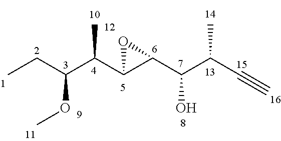 [SMILES: C#C[C@@H](C)[C@H](O)[C@H]1O[C@@H]1[C@H](C)[C@H](CC)OC]
the composition having greater than 99% entantiopurity at carbon 7 of the formula;
and
2) optionally, wherein the composition comprises less than 0.5 ppm Sn other than Sn in the formula.

14. A composition, comprising:
1) a compound of a formula selected from: 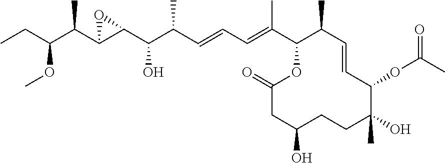 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]
the composition having the formula in greater than 99% entantiopurity, 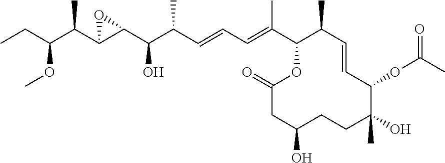 [SMILES: CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C]
the composition having the formula in greater than 99% entantiopurity, 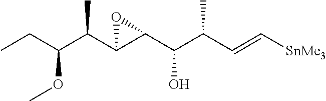
the composition having the formula in greater than 99% entantiopurity, 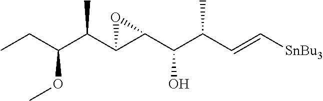
the composition having the formula in greater than 99% entantiopurity, 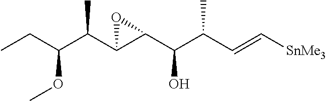
the composition having the formula in greater than 99% entantiopurity, 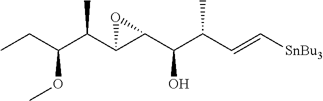
the composition having the formula in greater than 99% entantiopurity, 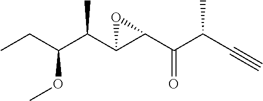 [SMILES: C#C[C@@H](C)C(=O)[C@H]1O[C@@H]1[C@H](C)[C@H](CC)OC]
the composition having the formula in greater than 99% entantiopurity, 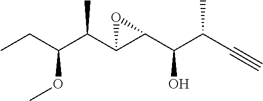 [SMILES: C#C[C@@H](C)[C@@H](O)[C@H]1O[C@@H]1[C@H](C)[C@H](CC)OC]
the composition having the formula in greater than 99% entantiopurity, or 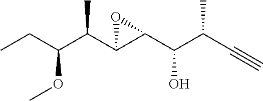 [SMILES: C#C[C@@H](C)[C@H](O)[C@H]1O[C@@H]1[C@H](C)[C@H](CC)OC]
the composition having the formula in greater than 99% entantiopurity; and
2) optionally, wherein the composition comprises less than 0.5 ppm Sn other than Sn in the formula.

15. A method of treating a neoplasm in a subject in need thereof, comprising administering a therapeutically effective amount of the compound of claim 12, or the composition of claim 13 or claim 14, to the subject.

16. A method of modulating spliceosomal activity in a cell, comprising contacting the cell with an effective amount of the compound of claim 12, or the composition of claim 13 or claim 14.

17. A method of modulating adenosine deaminase acting on RNA (ADAR) activity in a cell, comprising contacting the cell with an effective amount of the compound of claim 12, or the composition of claim 13 or claim 14.

18. A method, comprising a synthesis of Compound 19, wherein the synthesis comprises: contacting the compound of claim 2, which is Compound 190, 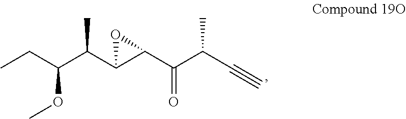 [SMILES: C#C[C@@H](C)C(=O)[C@H]1O[C@@H]1[C@H](C)[C@H](CC)OC]
with an enzyme having aldo-keto reductase activity, to form Compound 19,

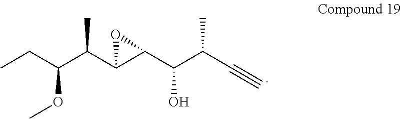 [SMILES: C#C[C@@H](C)[C@H](O)[C@H]1O[C@@H]1[C@H](C)[C@H](CC)OC]

19. A method, comprising a synthesis of Compound 7, wherein the synthesis comprises: contacting the compound of claim 2, which is Compound 6, 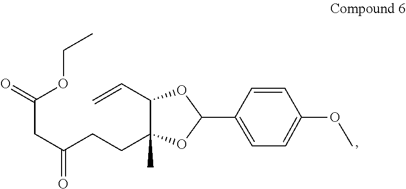
with an enzyme having aldo-keto reductase activity, to form Compound 7,

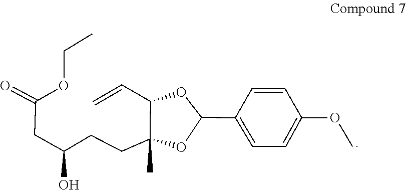

20. The method of claim 18 or claim 19, which is a method of preparing Compound A, 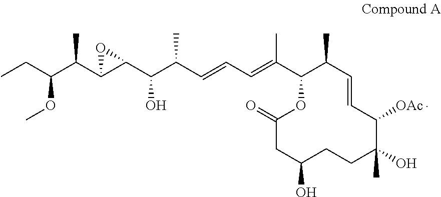 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]
